(19) United States
(12) Patent Application Publication
(10) Pub. No.: US 2025/0262429 A1
(43) Pub. Date: Aug. 21, 2025

(54) APPARATUS AND METHOD FOR CAUSING VASOCONSTRICTION BY ELECTRICAL STIMULATION

(71) Applicant: GUY'S AND ST THOMAS' NHS FOUNDATION TRUST, London (GB)

(72) Inventors: Emmanuel Adeoluwa AKINLUYI, London (GB); Sabrina RAMNARINE, London (GB); Charlotte Deirdre JONES, London (GB)

(21) Appl. No.: 18/867,367

(22) PCT Filed: May 19, 2023
(86) PCT No.: PCT/EP2023/063530

§ 371 Date: Nov. 19, 2024

(30) Foreign Application Priority Data
May 19, 2022 (GB) 2207368.8

(51) Int. Cl. A61N 1/04; A61N 1/36

(52) U.S. Cl. CPC A61N 1/0456; A61N 1/0484; A61N 1/36031

(57) ABSTRACT

An apparatus configured to be worn on the hand or foot, comprising: a plurality of electrodes arranged so as to be positioned over one or more respective phalanges when the apparatus is attached to the hand or foot; a supporting structure configured to attach the apparatus to the hand or foot and to retain the plurality of electrodes; and a controller configured to control the application of signals to the plurality of electrodes. Feedback sensors may be provided and positioned on a digits to monitor a property of underlying tissue.

### FIELD OF THE INVENTION

The present invention relates to a wearable apparatus configured to apply electrical stimulation to the autonomous (sympathetic) nervous system, to achieve targeted vasoconstriction.

### BACKGROUND

In the field of chemotherapy treatment, one of the side effects widely observed is Chemotherapy-Induced Peripheral Neuropathy (CIPN). Peripheral neuropathy may manifest as a tingling or numbness in the hands and feet, pain in the hands and feet and loss of function post chemotherapy leading to a deterioration in quality of life.

Toxicity from the chemotherapy medication is thought to cause nerve damage, leading to CIPN. This is a cumulative effect as the toxicity levels increase with each chemotherapy treatment. This can often lead to the reduction or cessation of chemotherapy, resulting in poorer therapeutic outcomes. CIPN is thought to affect the majority of patients undergoing chemotherapy treatment meaning that addressing the condition is an urgent unmet clinical need.

Current methods for treating CIPN are not well tolerated by patients and are not easy for healthcare professionals to apply. One existing solution is cold therapy to cause vasoconstriction. However, such devices have poor patient comfort and tolerability due to the very low temperatures involved. Due to the length of some chemotherapy sessions, a cold insert must be changed regularly, or several devices are required, which is time intensive for healthcare professionals. Such devices have also been known to cause thermal injuries such as frostbite.

### SUMMARY OF THE INVENTION

According to a first aspect of the invention, there is described an apparatus configured to be worn on the hand or foot, the apparatus comprising: a plurality of electrodes arranged so as to be positioned over one or more respective phalanges when the apparatus is attached to the hand or foot; a supporting structure configured to attach the apparatus to the hand or foot and to retain the plurality of electrodes; and a controller configured to control the application of signals to the plurality of electrodes.

Each of the plurality of electrodes may be arranged so as to be positioned over the proximal phalanx of a respective digit. Alternatively, each of the plurality of electrodes may be arranged so as to be positioned over the intermediate phalanx of a respective digit. Alternatively, each of the plurality of electrodes may be arranged so as to be positioned over the distal phalanx of a respective digit. Alternatively, each of the plurality of electrodes may be arranged so as to be positioned over the proximal and intermediate phalanges of a respective digit. Alternatively, each of the plurality of electrodes may be arranged so as to be positioned over all of the phalanges of a respective digit. The apparatus may further comprise one or more reference electrodes positioned on the hand or foot, away from the fingers or toes. The one or more reference electrodes may be positioned on the palm of the hand. The one or more reference electrodes may be positioned on the back of the hand. The one or more reference electrodes may be positioned on the sole of the foot. The one or more reference electrodes may be positioned on the top of the foot.

The apparatus may further comprise a sensor retained by the supporting structure, wherein the sensor is positioned on a digit and is configured to monitor a property of underlying tissue.

The sensor may comprise a photoplethysmography sensor configured to output signals to the controller. The controller may be configured to derive a peripheral perfusion index in the underlying tissue based on the signals.

The sensor may comprise a peripheral autonomic surface potential sensor configured to measure activity of autonomic nervous tissue underlying the sensor.

The sensor may comprises a surface electromyography sensor configured to measure activity of somatic nervous tissue underlying the sensor.

The controller may be configured to dynamically adjust a property of the signals applied to the plurality of electrodes based on signals received from the sensor.

The apparatus may comprise a plurality of sensors, each of the plurality of sensors positioned on a respective digit. Alternatively, some subset of the digits may have corresponding sensors.

The controller may be configured to apply signals to the plurality of electrodes periodically, with a duty cycle of 50%.

The apparatus may further comprise a secondary electrode arranged to be positioned on the forearm or foreleg when the apparatus is attached to the hand or foot. The secondary electrode may be positioned so as to provide an analgesic effect when a stimulus is applied.

The apparatus may comprise a garment configured to be worn on the hand and optionally wherein the garment is a glove or mitt. Configuring the garment as a glove may be advantageous, as the patient retains some degree of dexterity while the electrical stimulus is being applied and can therefore continue to perform some tasks during the treatment.

The apparatus may comprise a garment configured to be worn on the foot and optionally wherein the garment is a sock or shoe.

According to a second aspect of the invention, there is described a method of inducing vasoconstriction in peripheral nerves using electrical stimulation, the method comprising: attaching an apparatus comprising a plurality of electrodes to a hand or foot of a patient, such that each electrode is positioned over one or more respective phalanges of the patient; applying a series of test signals with the plurality of electrodes to calibrate a threshold of perception at which the patient perceives the test signals; and applying treatment signals with the plurality of electrodes, the treatment signals being below the threshold of perception established for the patient.

The method may further comprise: monitoring a level of vasoconstriction using one or more sensors; and adjusting one or more parameters of the treatment signals based on the monitored level of vasoconstriction.

The apparatus may comprise a secondary electrode arranged to be positioned on the forearm or foreleg when the apparatus is attached to the hand or foot of the patient, and the method may further comprise subsequent to applying the treatment signals, applying one or more secondary signals with the secondary electrode to induce an analgesic and/or massage effect on the underlying tissue.

### BRIEF DESCRIPTION OF THE FIGURES

So that the general concepts set out in the foregoing sections can be more fully understood, embodiments thereof will be described with reference to the accompanying drawings, in which:

FIG. 1 shows an apparatus having the form of a glove with an integrated control system;

FIG. 2 shows a system according to some other embodiments, in which a control system is located externally to the apparatus;

FIG. 3 shows an apparatus according to further embodiments comprising monitoring sensors;

FIG. 4a is a graph showing results from a prototype apparatus in which a single electrode was attached to the fifth finger;

FIG. 4b shows a series of graphs showing experimental results with electrodes positioned over the proximal phalanges;

FIG. 4c shows a series of graphs showing experimental results in which different positions of the electrodes were tested;

FIG. 5 shows an apparatus according to further embodiments comprising elongated electrodes and an optional secondary electrode;

FIG. 6 shows an apparatus according to further embodiments in which the end portions of the fingers sections are omitted;

FIG. 7 illustrates schematically an electronic system for controlling operation of the apparatus of any of FIGS. 1-3, 5-6 and 8;

FIG. 8 shows an apparatus according to further embodiments having the form of a sock; and

FIG. 9 is a flow chart illustrating an exemplary method for applying a treatment for inducing vasoconstriction in peripheral nerves using electrical stimulation.

### DETAILED DESCRIPTION

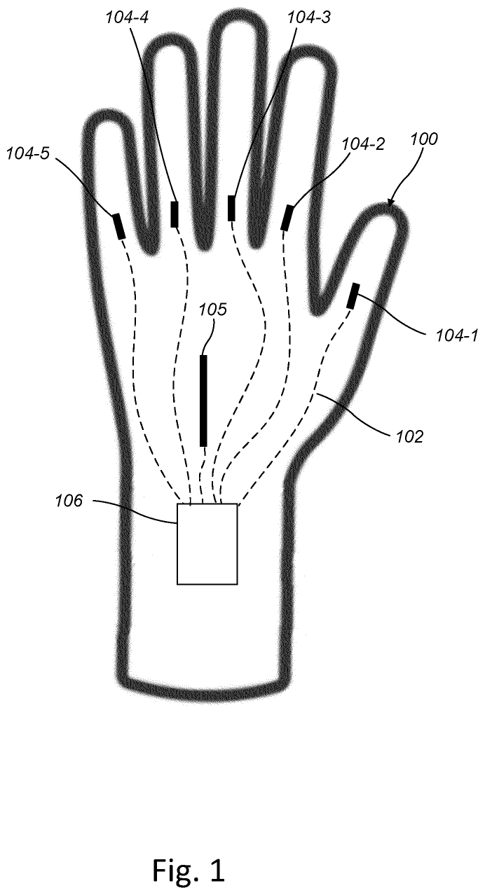

Referring to FIG. 1, an apparatus 100 is shown having the form of a glove. The apparatus 100 comprises a supporting structure 102 allowing it to be attached to the hand of the user. The apparatus 100 also comprises a plurality of electrodes 104-1 to 104-5 (generally denoted as 104). Each electrode 104 is located in a respective finger of the glove so as to be positioned on a respective finger of the user when the user is wearing the glove. The electrodes 104 may be positioned on the upper side of each glove finger so as to contact an upper surface of each finger, or on a lower side of each glove finger so as to contact an lower surface of each finger. Alternatively, each electrode 104 may have a ring or partial ring shape so as to wrap partially or completely around the respective finger.

The electrodes 104 may extend longitudinally along the length of the user's finger, as shown in FIG. 1. Alternatively, the electrodes 104 may be positioned transverse to the user's fingers. In such an arrangement, each electrode 104 may wrap partially around the respective finger so as to cover both the medial and lateral sides of the finger.

In the embodiment shown in FIG. 1, the electrodes 104-1 to 104-5 are each positioned on a proximal phalanx of the respective finger. In some other embodiments, described below, the electrodes 104-1 to 104-5 may be positions on the intermediate or distal phalanges or extend over two or more phalanges.

In addition to these, a reference electrode 105 is placed on/around the palm or wrist. Each of the electrodes 104-1 to 104-5 may share a common reference electrode 105. Alternatively, multiple reference electrodes 105 may be provided, such that each electrode 104 has a corresponding reference electrode 105. The reference electrode 105 may extend longitudinally along the length of the user's palm, as shown in FIG. 1. Alternatively, the reference electrode 105 may be positioned across the user's palm (transverse to the direction of the fingers). In general, the reference electrode(s) 105 is positioned away from the fingers.

Placing the reference electrode 105 on the user's palm has been observed to have the greatest vasoconstriction effect. However, the reference electrode 105 may alternatively be positions on the back of the user's hand.

The electrodes 104-1 to 104-5 may be the anodes of the system while the reference electrode may be the cathode. With this arrangement, the signals applies to the electrodes 104 stimulate along the user's fingers.

The apparatus 100 also comprises a control system 106, which in this embodiment is supported on a wrist portion of the glove.

The supporting structure 102 may be a glove with modified regions for retaining the electrodes 104, and an elongated wrist portion with a modified region or pocket for retaining the control system 106. The supporting structure 102 may comprise holes or other access points to allow adjustment or replacement of each electrode 104. In some other embodiments, the supporting structure 102 may comprise a frame or web structure. This may allow increased airflow to the hand compared with a glove. The electrodes 104, 105 and control system 106 may be retained by adjustable and/or resilient straps. Alternatively, or in addition, the electrodes may have a sticky portion for securing them to the user's skin.

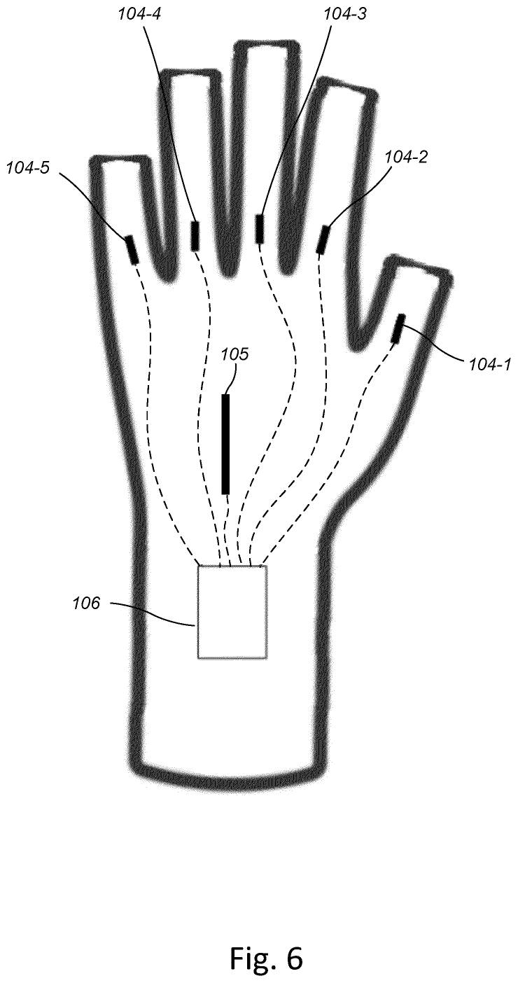

The control system 106 may comprise a power supply, pulse generator and a processing unit (optionally incorporating feedback circuitry) for controlling the application of electrical signals to the electrodes 104. Each of the electrodes 104 may have a separate connection to the control system 106 for this purpose. In some embodiments, the reference electrode may be located directly underneath the control system and/or may be integral with the control system. The control system may also comprise further elements described in more detail with respect to FIG. 6.

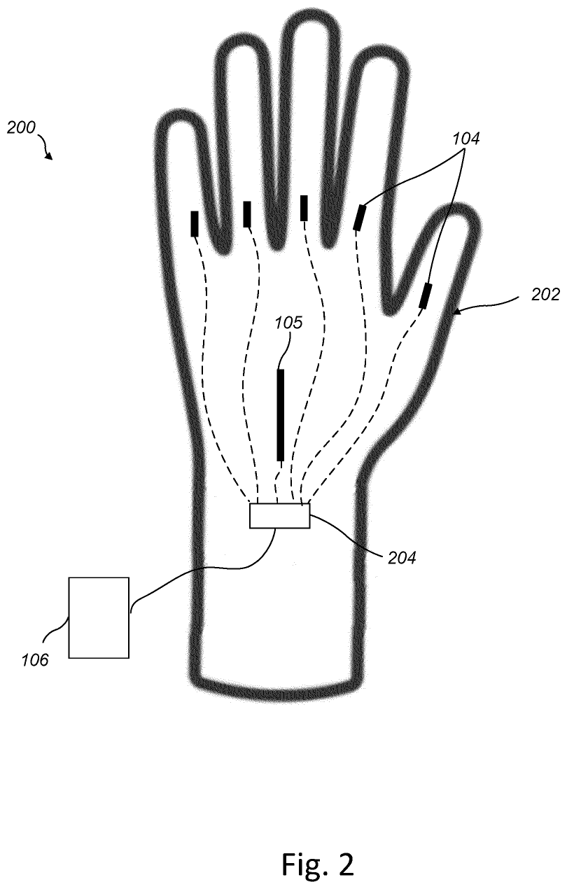

FIG. 2 shows a system 200 according to some other embodiments, in which the control system 106 is located externally to the apparatus 202. The apparatus 202 comprises a hub 204 into which all of the electrodes 104 and the reference electrode 105 are connected. The hub 204 can in turn be connected and disconnected from the control system 106 via a port on the hub 204 and/or control system 106. This arrangement reduces the complexity of the apparatus 202 and allows the control system 106 to be used with different apparatuses 202, meaning that fewer control systems 106 are required.

In both of the embodiments of FIGS. 1 and 2, the electronic parts of the apparatus 100, 202 may be removable from the supporting structure 102, which allows the supporting structure to be washed. Alternatively, some or all of the electronic parts may be sown into or otherwise permanently attached to the supporting structure 102. The supporting structure 102 may be made of a resilient material to allow it to be used on patients having different sized hands. Alternatively, or in addition, the apparatus 100, 202 may come in a variety of different sizes.

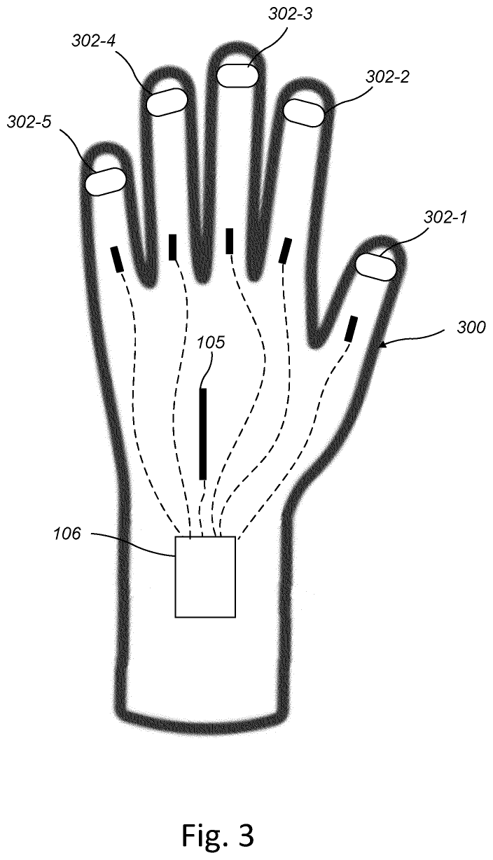

Referring to FIG. 3, an apparatus 300 is shown according to further embodiments. The apparatus 300 comprises the same arrangement of electrodes 104, reference electrode 105 and control system 106 disclosed in the embodiment of FIG. 1. In addition, the apparatus 300 comprises at least one sensor 302-1 to 302-5 (generally denoted as 302) retained by the supporting structure 102. Each sensor 302 is arranged so as to be positioned on a digit and is configured to monitor a property of underlying tissue.

Although FIG. 3 illustrates a sensor 302 positioned on each finger, in some embodiments, only a subset of the illustrated sensors may be present. In some embodiments only a single sensor may be used. A number of different sensing technologies may be employed for the sensors 302. In some embodiments the sensors may comprise photoplethysmography (PPG) sensors configured to output signals to the controller system 106. The PPG sensors are positioned on the lower side of the glove so as to be in contact with the pad of each finger when the patient is wearing the glove. The control system 106 is configured to receive the signals from the PPG sensors and to derive an indication of peripheral perfusion (such as the peripheral perfusion index—PPI) in the underlying tissue based on the received optical signals. The PPI gives a measure of blood flow in the finger.

In some other embodiments, the sensors comprise peripheral autonomic surface potential sensors. These are configured to measure an electric potential which is indicative of the activity of autonomic nervous tissue underlying the sensor. The control system 106 receives signals from the peripheral autonomic surface potential sensors and is configured to determine a measure of blood flow in the finger.

In some other embodiments, the sensors comprise surface electromyography sensors. These are configured to measure activity of somatic nervous tissue underlying the sensor.

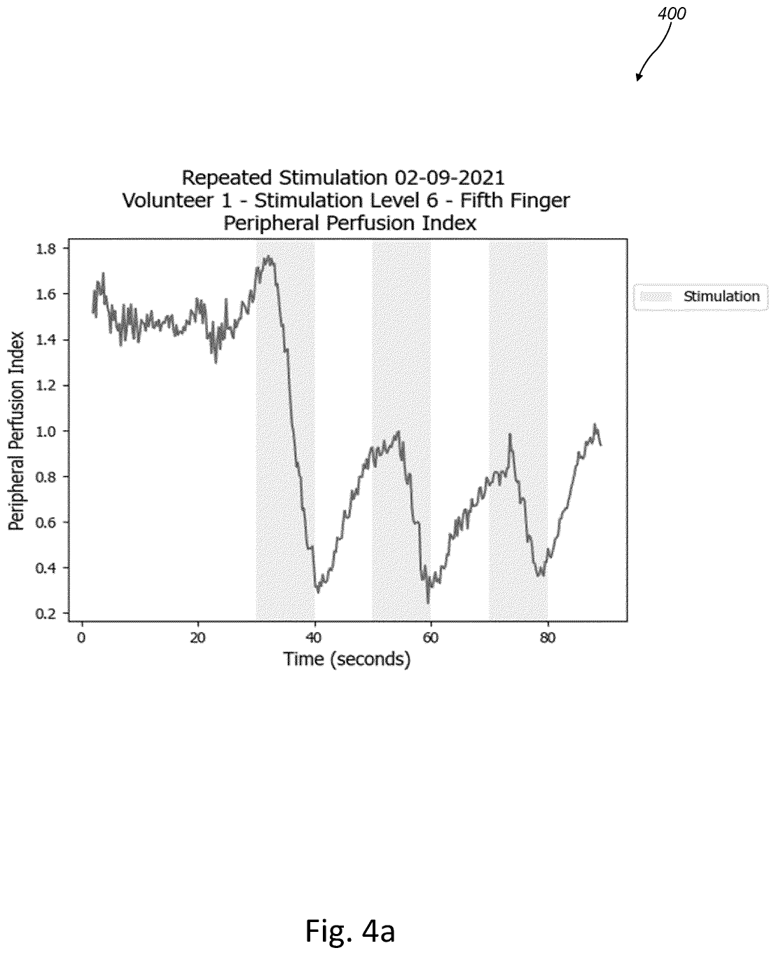

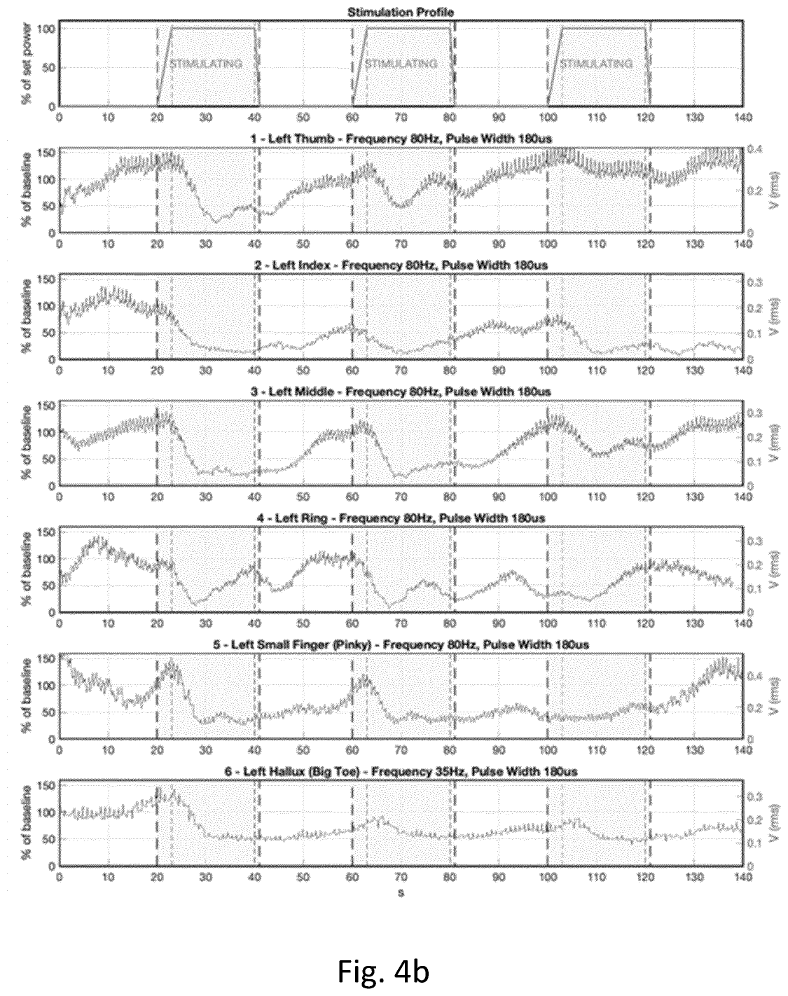

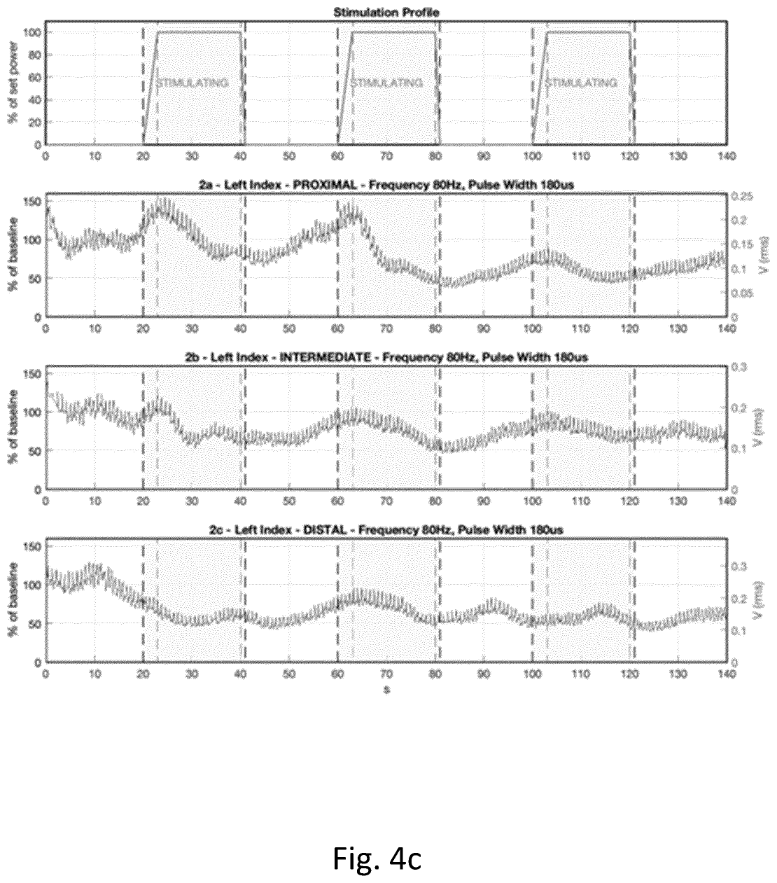

In some embodiments, the electrodes 104 used to apply a stimulus to the fingers may also be used as sensors. In the embodiments described above, the peripheral autonomic surface potential sensors or surface electromyography sensors comprises electrodes. These may be the same electrodes 104 that are used to stimulate the fingers. As described below with reference to FIG. 4, the stimulus is applied periodically. During the periods where no signal is applied to the electrodes 104, they may be used as sensors.

Measuring the current blood flow in the fingers as the electrical stimuli are applied to the electrodes allows the effectiveness of the treatment to be monitored in real time. The information from the sensors can also be used as a feedback mechanism to dynamically alter the strength, frequency and/or periodicity of the signals being applied to the electrodes to achieve the highest ongoing levels of vasoconstriction during a chemotherapy session.

FIG. 4a is a graph 400 showing results from a prototype apparatus in which a single electrode was attached to the fifth finger, with a reference electrode attached to the palm. The electrode extended over both the proximal and intermediate phalanges. A PPG sensor was placed on the finger tip of the fifth finger. Values for peripheral perfusion index were derived from the optical signals received from the PPG sensor during the test. An acclimatization period of 30 seconds was given before the application of any signal. Then an electrical stimulus was applied for 10 seconds, followed by an off period of 10 seconds, repeated periodically. The frequency of the applied signal was ˜50 hz, with a current of 15mA and pulse width ˜100 μs.

As can be seen, during the initial period of electrical stimulation the PPI fell sharply down to approximately 20% of the pre stimulus value. When the stimulus was removed for, the PPI rose over the period of no stimulus, but dropped again when the stimulus was re-applied. As can be seen from the graph 400, applying the electrical stimulus periodically for 10 seconds, with a duty cycle of 50%, resulted in an average PPI over the period of testing of approximately 40% of the pre-stimulus value. This result indicates that substantial vasoconstriction can be achieved by the targeted application of periodic electrical signals on the phalanges. The likely mechanism is by the inhibition of local nitric oxide release, and therefore the activation of a local autonomic (sympathetic) response.

FIG. 4b contains a series of graphs showing firstly a stimulation profile and then a number of experimental results of root-mean-square PPG signals against time. These results were obtained with an apparatus as shown in FIG. 3, with electrodes 104 positioned on the proximal phalanges of the fingers and a reference electrode positioned on the palm. PPG sensors 302 were positioned in contact with the pad of each finger. After attaching the apparatus 300 to the user, an extended settling period occurred in which no signals were applied.

The electrical stimulus was then applied periodically for 20 seconds, with a duty cycle of 50%. The frequency of the applied signal was ˜80 hz, with a pulse width ˜180 μs. The first 20 seconds of the measurement period, in which no signal was applied, was used as a baseline, against which the signal is normalized in plotting, allowing for comparison of the response between fingers. As can be seen in the first plot in FIG. 4b, when beginning or resuming stimulation, the apparatus has a linear ramp up over a period of 3 s. On pausing, the apparatus would cease stimulation via a linear ramp down over a period of 1 s.

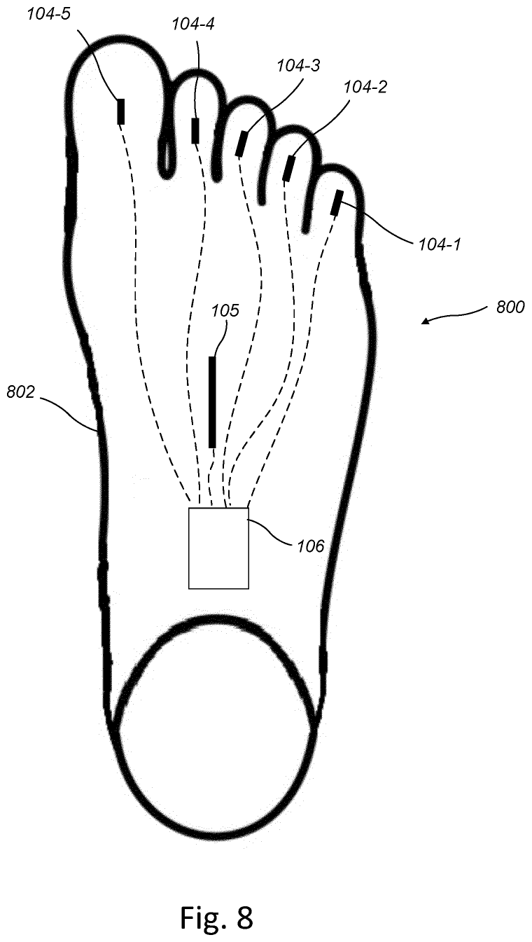

Six different configurations are then shown, representing results from the thumb, index, middle, ring and small fingers of the left hand of the user. The last configuration shows results from the hallux (big toe) of a user (see description of FIG. 8 below). As can be seen there is a significant drop in PPG signal strength when the stimulation signal is applied, caused by substantial vasoconstriction in the peripheral nerves of the finger pads. These tests demonstrate a clear ability to induce vasoconstriction and to maintain this reduced blood flow over a prolonged period.

During these tests, the inventors noted difficulties in retaining the PPG probe against the thumb as it deforms, as is evident in the progressive degradation of the detected vasoconstrictive effect in configuration 1.

FIG. 4c contains a series of graphs showing firstly a stimulation profile and then a number of experimental results of root-mean-square PPG signals against time, in which different positions of the electrodes 104 were tested. The stimulation profile is the same as that described with reference to FIG. 4b. After attaching the apparatus to the user, an extended settling period also occurred in which no signals were applied. The first 20 seconds of the measurement period, in which no signal was applied, was again used as a baseline, against which the signal is normalized in plotting.

Configuration 2a in FIG. 4c comprised an electrode positioned on the proximal phalanx of the left index finger, with a reference electrode positioned on the palm and a PPG sensor on the left index finger pad. Configuration 2b in FIG. 4c comprised an electrode positioned on the intermediate phalanx of the left index finger, with a reference electrode positioned on the palm and a PPG sensor on the left index finger pad. Configuration 2c in FIG. 4c comprised an electrode positioned on the distal phalanx of the left index finger, with a reference electrode positioned on the palm and a PPG sensor on the left index finger pad.

As can be seen, the largest drop in PPG signal during stimulation was observed when the electrode is positioned over the proximal phalanx, and in general the effect appears more pronounced with stimulation at the proximal and intermediate phalanges. However, in all cases a sustained long term reduction in PPG signal is observed, resulting from a sustained vasoconstriction and reduction in blood flow in the finger tips.

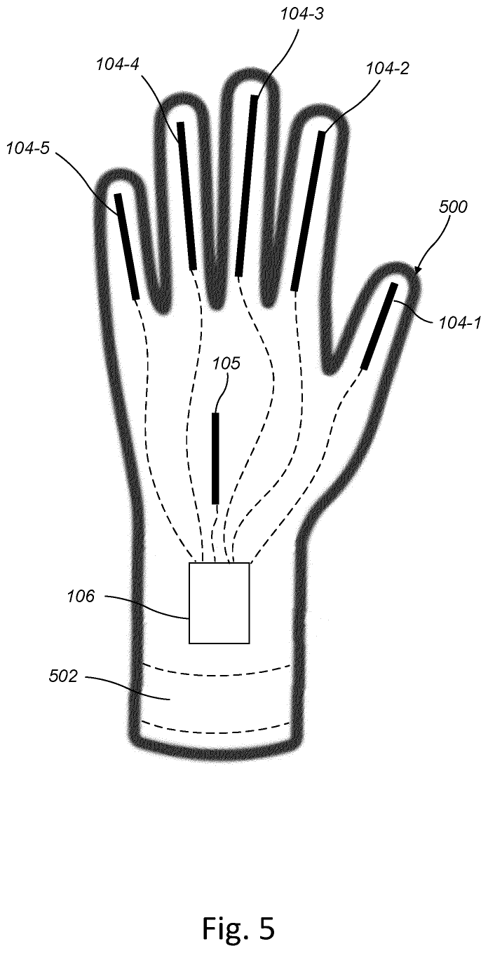

Referring to FIG. 5, an apparatus 500 is shown according to further embodiments. The apparatus 500 comprises elongated electrodes 104 which extend along all of the phalanges of the fingers. The electrodes 104 are otherwise the same as those previously described. A reference electrode 105 is position on the palm (or back of the hand) as previously described. The electrodes are connected to and controlled by a control system 106 similar to that already described. In addition, the apparatus 500 may optionally comprise a secondary electrode 502 supported on the elongated wrist portion of the glove, so as to be positioned on the forearm of the patient when the patient is wearing the glove. The position of the secondary electrode 502 is such that applying a stimulus will result in vasodilation of the underlying tissue. The secondary electrode may be a standard TENS electrode. As such, the secondary electrode 502 is not used during a chemotherapy session, but may be activated post therapy for the purpose of increasing peripheral blood-flow, causing an analgaesic effect or massage effect.

Referring to FIG. 6, an apparatus 600 is shown according to further embodiments. The apparatus 600 comprises the same arrangement of electrodes 104, reference electrode 105 and control system 106 disclosed in the embodiment of FIG. 1. However, the end portions of the fingers of the glove are removed, so that the patient's finger tips will be exposed when they are wearing the apparatus 600. This allows the apparatus 600 to be used in combination with an existing PPI monitor such as a pulse oximeter. This design may also allow the patient greater dexterity to carry out tasks while undergoing a chemotherapy treatment.

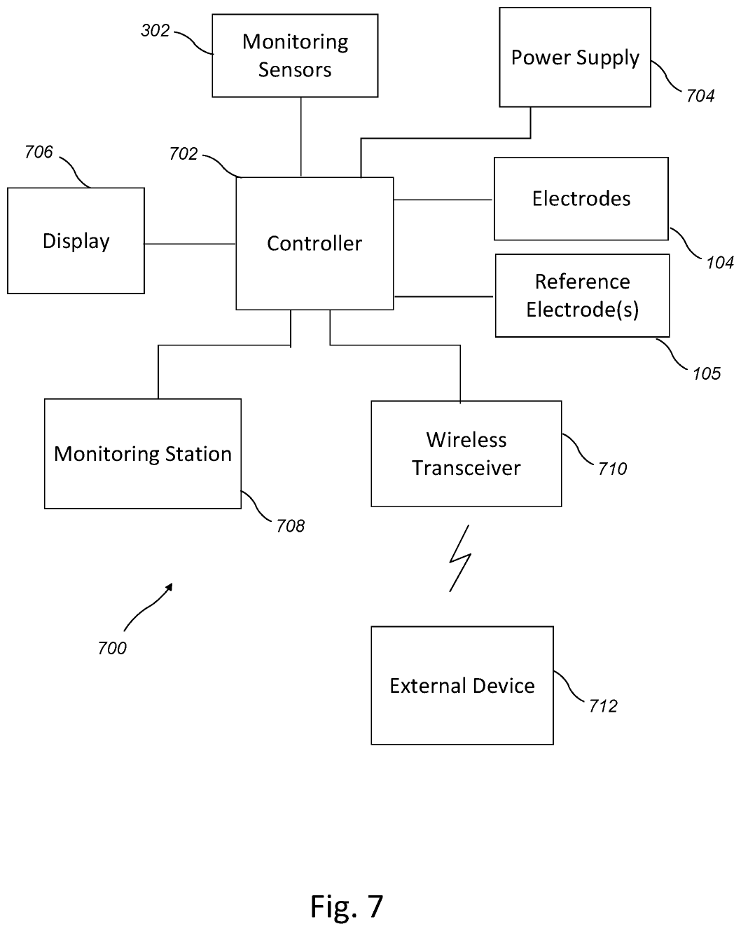

FIG. 7 illustrates schematically an electronic system 700 for controlling operation of the previously described apparatus. The electronic system 700 comprises a controller 702, the previously described electrodes 104, reference electrode(s) 105, one or more of the previously described monitoring sensors 302 and a power supply 704. Optionally, the electronic system may also include a display 706, monitoring station 708 and/or wireless transceiver 710.

The controller 702 is configured to control operation of the other components of the electronic system 700. The controller 702 may for instance be a microprocessor, a Digital Signal Processor (DSP), Application Specific Integrated Circuit (ASIC), Field Programmable Gate Array (FPGA) or the like. Alternatively, the controller 702 may comprise specialized processing hardware, for instance a RISC processor or programmable hardware with embedded firmware. The controller 702 may comprise multiple processors. The controller 702 may also comprise a memory, for example a program memory storing program code (e.g. software or firmware). The program memory may for instance be a non-volatile memory, such as a Read-Only Memory (ROM), a Flash memory or a magnetic drive memory.

The display 706 may be a standard LCD display or a touch sensitive display based on capacitive or resistive sensing technology. The display 706 may be attached, either removably or permanently, to the supporting structure 102 of the apparatus. Where the apparatus is a glove, the display 706 may be positioned on the elongated wrist portion of the glove, so that it can be easily viewed by the patient, without interfering with either the chemotherapy treatment or the electrical stimulus being provided by the apparatus. The power supply 704 may be a battery of any suitable kind and may provide power for all of the other elements of the electronic system 700. The power supply 704 may also be retained on or in the supporting structure 102.

The electronic system 700 may also optionally comprise a wireless transceiver 710 for communicating with an external device 712. The wireless transceiver 710 comprises the network interfaces necessary to communicate over 3G, 4G, 5G, Bluetooth, WiFi, Zigbee or any combination of these protocols and may also include an antenna. The wireless transceiver 710 may communicate directly with the external device 712, or the communication may be routed through an intermediate device, router, server or base station. The external device 712 may be a smartphone, tablet, PDA or other mobile computing device. The external device 712 may be assigned to the patient or the patient's doctor or other health care professional. The external device 712 may run an app or other software to display information relating to the operation of the electronic system 700. Further information about the patient's chemotherapy may be integrated into the app, such as the duration of the current treatment, time remaining. Information received from the monitoring sensors 302 may be displayed on a display of the external device 712. The app or other software on the external device 712 may also allow for control of the electronic system 700, for example to start, stop and/or pause the electrical stimulus or change a parameter of the applied signals, such as the current or periodicity (duty cycle). The external device 712 also comprises a memory (not shown) which may be used to record any information received from the monitoring sensors 302 for subsequent review or onward transmission.

In some embodiments, the controller 702 and power supply 704 may be incorporated in the control system 106 shown in FIGS. 1, 2, 5 and 6. The display 706 and wireless transceiver 710, if present, may also be part of the control system 106. The electrodes 104 and monitoring sensors 302 (if present) are necessarily remote from the control system 106, so that these components can be located on the user's fingers.

In some other embodiments, the electronic system 700 comprises a monitoring station 708, to which the controller 702 can be connected. The monitoring station 708 is external to the apparatus and may comprise a computer terminal. The monitoring station 708 may receive information from the monitoring sensors 302 while the apparatus is in use. This may allow a doctor, nurse or other healthcare professional to observe the effects of the electrical stimulus in real time. In some embodiments, the monitoring station 708 may be capable of issuing instructions to the controller 702 to alter a parameter of the applied signals, such as the current or periodicity (duty cycle). This may be done automatically as a result of feedback from the monitoring sensors 302, in order to maximize the reduction in peripheral blood flow, or to keep the peripheral blood flow at or within defined limits.

In use the controller 702 controls application of electrical signals by the power supply 704 to the electrodes 104, according to a pre-set program. The pre-set program may be stored in a memory on the controller, a separate memory (not shown) forming part of the electronic system 700 or on the monitoring station 708.

The controller 702 receives feedback signals from the monitoring sensors 302 and controls display of this information on the display 706, if present. The feedback information may be communicated to the monitoring station 708 for display and/or storage. The feedback information may also be communicated via the wireless transceiver 710 to the external device 712, for display and/or storage. The controller 702 may receive instructions from the monitoring station 708 or the external device 712 to alter one or more parameters of the applied signals.

Referring to FIG. 8, an apparatus 800 is shown according to further embodiments. The apparatus 800 comprises a sock or other garment arrangement to be worn on the foot of a patient. The apparatus 800 comprises a supporting structure 802 allowing it to be attached to the foot of the user.

The apparatus 800 comprises a plurality of electrodes 104. Each electrode 104 is located in a respective toe of the sock so as to be positioned on a respective toe of the user when the user is wearing the sock. The electrodes 104 may be positioned on the upper side of each toe area so as to contact an upper surface of each toe, or on a lower side of each toe area so as to contact an lower surface of each toe. Alternatively, each electrode 104 may have a ring or partial ring shape so as to wrap partially or completely around the respective toe. In addition to these, a reference electrode 105 is placed on/around the sole of the foot. In some alternative embodiments, the reference electrode 105 is positioned on the top of the foot part of the sock or on the ankle part of the sock. In general, the reference electrode(s) 105 is positioned away from the toes.

The apparatus 800 also comprises the control system 106 as previously described, which in this embodiment is supported on the top of the foot part of the sock. The control system 106 in FIG. 8 may comprise some of the elements of the electronic system 700 as previously described and may be able to be connected to a monitoring station 708 and/or communicate with an external device 712, as previously described.

The supporting structure 802 may be a sock with modified regions for retaining the electrodes 104. The supporting structure 802 may comprise holes or other access points to allow adjustment or replacement of each electrode 104.

As with the glove embodiments described, above, the electrodes 104 in the sock embodiment of FIG. 8 are positioned so as to apply electrical stimulation directly to the phalanges, in this case the phalanges of the toes. Application of an electrical signal in this manner results in vasoconstriction of the blood vessels in the toes, which reduces the build up of toxicity as a result of chemotherapy medication in the toes and a resulting reduction in nerve damage. The electrodes 104 may also act as sensors for determining activity of the underlying nerve tissue in order to monitor the effectiveness of the electrical stimulation and to act as a feedback for altering the electrical stimulus if necessary. In some other embodiment, one or more of the toe sections of the apparatus 800 may additionally be provided with a PPG sensor to independently monitor blood flow and allow a PPI to be derived. Alternatively, the end portions of one or more of the toe section may be omitted to allow a separate PPG sensor to be attached to the toe.

The apparatus 800 may also comprise a secondary electrode (not shown) supported by an elongated ankle portion (not shown) of the sock, such that the secondary electrode is positioned on the foreleg of the patient. As previously described, the secondary electrode is not used during a chemotherapy session, but may be employed post therapy to provide an analgesic or massage effect.

Although the apparatus 800 has been shown with separate section for each toe, the apparatus 800 may instead comprise a single internal space, with the electrodes 104 positioned so that they can be accurately placed on the toes of the patient. Additionally, although he apparatus 800 is depicted as a sock with separate sections for each toe, the apparatus may instead take the form of a slipper or shoe.

Referring again to FIG. 4b, experimental results obtained by applying a signal to the hallux (big toe) are shown in chart 6. In this experiment, the electrode was positioned on the proximal phalanx of the big toe and a PPG sensor attached to the toe pad. As can be seen, a significant and sustained vasoconstriction can be obtained in the toe using this apparatus.

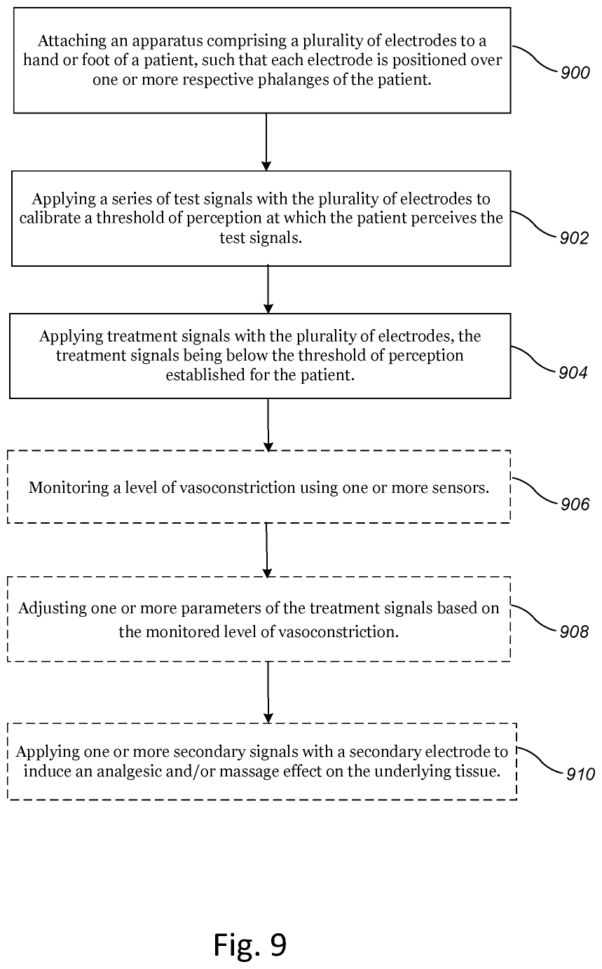

FIG. 9 is a flow chart illustrating an exemplary method for applying a treatment for inducing vasoconstriction in peripheral nerves using electrical stimulation. The treatment may be applied simultaneously with a chemotherapy sessions, i.e. during administration of a chemotherapy medication, for the purpose of reducing the accumulation of toxicity in peripheral nerves during chemotherapy, which can lead to peripheral neuropathy.

The process begins at step 900, by attaching an apparatus comprising a plurality of electrodes to a hand or foot of a patient, such that each electrode is positioned over one or more respective phalanges of the patient. The apparatus may be any of those described with reference to FIG. 1-3, 5, 6 or 8.

At step 902 a series of test signals are applied with the plurality of electrodes to calibrate a threshold of perception at which the patient perceives the test signals. Ideally, the level of electrical signal used during treatment will be mostly imperceptible to the user, both for increased comfort and for improved uptake of the treatment. Each patient will have a different perception threshold and so it is necessary to establish a calibrated perception level for each patient before treatment.

At step 904, treatment signals are applied with the plurality of electrodes, the treatment signals being below the threshold of perception established for the patient.

At optional further step 906, one or more sensors are used to monitor a level of vasoconstriction. These sensor may be any of the sensors 302 previously described. At optional further step 908, one or more parameters of the treatment signals are adjusted based on the monitored level of vasoconstriction. The adjustments to the treatment signals are made within predefined limits and also take into account the perception threshold of the individual patient. At optional further step 910, one or more secondary signals are applied with a secondary electrode to induce an analgesic and/or massage effect on the underlying tissue. This step is performed after the treatment signals are completed.

### REFERENCE NUMERALS

100 Apparatus
102 supporting structure
104 electrodes
104-1 first electrode etc.
105 reference electrode
106 control system
200 System
202 Apparatus
204 hub
300 apparatus
302 sensors
302-1 first sensor etc.
400 graph
500 Apparatus
502 Secondary electrode
600 apparatus
700 Electronic System
702 controller
704 power supply
706 display
708 monitoring station
710 wireless transceiver
712 external device
800 apparatus
802 supporting structure

1. An apparatus configured to be worn on the hand or foot, the apparatus comprising:
a plurality of electrodes arranged so as to be positioned over one or more respective phalanges when the apparatus is attached to the hand or foot;
a supporting structure configured to attach the apparatus to the hand or foot and to retain the plurality of electrodes; and
a controller configured to control the application of signals to the plurality of electrodes.

2. The apparatus of claim 1, wherein each of the plurality of electrodes is arranged so as to be positioned over the proximal phalanx of a respective digit.

3. The apparatus of claim 1, wherein each of the plurality of electrodes is arranged so as to be positioned over the intermediate phalanx of a respective digit.

4. The apparatus of claim 1, wherein each of the plurality of electrodes is arranged so as to be positioned over the distal phalanx of a respective digit.

5. The apparatus of claim 1, wherein each of the plurality of electrodes is arranged so as to be positioned over the proximal and intermediate phalanges of a respective digit.

6. The apparatus of claim 1, wherein each of the plurality of electrodes is arranged so as to be positioned over all of the phalanges of a respective digit.

7. The apparatus of any preceding claim, the apparatus further comprising one or more reference electrodes positioned on the hand or foot, away from the fingers or toes.

8. The apparatus of any preceding claim, the apparatus further comprising a sensor retained by the supporting structure, wherein the sensor is positioned on a digit and is configured to monitor a property of underlying tissue.

9. The apparatus of claim 8, wherein the sensor comprises a photoplethysmography sensor configured to output signals to the controller.

10. The apparatus of claim 9, wherein the controller is configured to derive a peripheral perfusion index in the underlying tissue based on the signals.

11. The apparatus of claim 8, wherein the sensor comprises a peripheral autonomic surface potential sensor configured to measure activity of autonomic nervous tissue underlying the sensor.

12. The apparatus of claim 8, wherein the sensor comprises a surface electromyography sensor configured to measure activity of somatic nervous tissue underlying the sensor.

13. The apparatus of any of claims 8 to 12, wherein the controller is configured to dynamically adjust a property of the signals applied to the plurality of electrodes based on signals received from the sensor.

14. The apparatus of any of claims 8 to 12, wherein the apparatus comprises a plurality of sensors, each of the plurality of sensors positioned on a respective digit.

15. The apparatus of any preceding claim, wherein the controller is configured to apply signals to the plurality of electrodes periodically, with a duty cycle of 50%.

16. The apparatus of any preceding claim, wherein the apparatus further comprises a secondary electrode arranged to be positioned on the forearm or foreleg when the apparatus is attached to the hand or foot.

17. The apparatus of any preceding claim, wherein the apparatus comprises a garment configured to be worn on the hand and optionally wherein the garment is a glove or mitt.

18. The apparatus of any of claims 1 to 16, wherein the apparatus comprises a garment configured to be worn on the foot and optionally wherein the garment is a sock or shoe.

19. A method of inducing vasoconstriction in peripheral nerves using electrical stimulation, the method comprising:
attaching an apparatus comprising a plurality of electrodes to a hand or foot of a patient, such that each electrode is positioned over one or more respective phalanges of the patient;
applying a series of test signals with the plurality of electrodes to calibrate a threshold of perception at which the patient perceives the test signals; and
applying treatment signals with the plurality of electrodes, the treatment signals being below the threshold of perception established for the patient.

20. The method of claim 19, further comprising:
monitoring a level of vasoconstriction using one or more sensors; and
adjusting one or more parameters of the treatment signals based on the monitored level of vasoconstriction.

21. The method of claim 19 or 20, wherein the apparatus comprises a secondary electrode arranged to be positioned on the forearm or foreleg when the apparatus is attached to the hand or foot of the patient, the method further comprising subsequent to applying the treatment signals, applying one or more secondary signals with the secondary electrode to induce an analgesic and/or massage effect on the underlying tissue.
